(19) United States
(12) Patent Application Publication
(10) Pub. No.: US 2024/0120024 A1
(43) Pub. Date: Apr. 11, 2024

(54) MACHINE LEARNING PIPELINE FOR GENOME-WIDE ASSOCIATION STUDIES

(71) Applicants: Illumina Software, Inc., San Diego, CA (US); Illumina, Inc., San Diego, CA (US); Illumina Australia Pty Ltd, Melbourne (AU); Illumina Netherlands BV, Eindhoven (NL); Illumina France SARL, Paris (FR); Illumina Cambridge Limited, Cambridge (GB)

(72) Inventors: Yair Field, Sunnyvale, CA (US); Jacob Christopher Ulirsch, Pacific Grove, CA (US); Cinzia Malangone, Cambridge (GB); Miguel Madrid-Mencia, Albi (FR); Geoffrey Nilsen, Palo Alto, CA (US); Pam Tang Cheng, Redwood City, CA (US); Ileena Mitra, San Jose, CA (US); Petko Plamenov Fiziev, Corona, CA (US); Sabrina Rashid, Beaverton, OR (US); Anthonius Petrus Nicolaas de Boer, Grass Valley, CA (US); Pierrick Wainschtein, Auchenflower (AU); Vlad Mihai Sima, Delft (NL); Francois Aguet, Foster City, CA (US); Kai-How Farh, Hillsborough, CA (US)

(21) Appl. No.: 18/483,313

(22) Filed: Oct. 9, 2023

Related U.S. Application Data
(60) Provisional application No. 63/415,534, filed on Oct. 12, 2022.
(60) Provisional application No. 63/378,873, filed on Oct. 9, 2022.

(51) Int. Cl. G16B 20/00; G16B 40/20

(52) U.S. Cl. CPC G16B 20/00; G16B 40/20

(57) ABSTRACT

Genome-wide association studies may allow for detection of variants that are statistically significantly associated with disease risk. However, inferring which are the genes underlying these variant associations may be difficult. The presently disclosed approaches utilize machine learning techniques to predict genes from genome-wide association study summary statistics that substantially improves causal gene identification in terms of both precision and recall compared to other techniques.

### CROSS REFERENCE TO RELATED APPLICATIONS

This application claims priority from and the benefit of U.S. Provisional Application Ser. No. 63/415,534, entitled “MACHINE LEARNING PIPELINE FOR GENOME-WIDE ASSOCIATION STUDIES”, filed Oct. 12, 2022, and U.S. Provisional Application Ser. No. 63/378,873, entitled “MACHINE LEARNING PIPELINE FOR GENOME-WIDE ASSOCIATION STUDIES”, filed Oct. 9, 2022, each of which is hereby incorporated by reference in its entirety for all purposes.

### FIELD OF THE TECHNOLOGY DISCLOSED

The technology disclosed relates to the use of machine learning (ML) techniques, which may be referred to as artificial intelligence, implemented on computers and digital data processing systems for the purpose of detecting causal genes underlying variant associations from genome-wide association studies (GWAS). The technology disclosed relates generally to using ML-based techniques for training, generating, or updating models for identifying such causal genes in large data sets, such as, but not limited to, genome-wide association studies, as well as the use or refinement of such models in causal gene identification.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Genetic variations can help explain many diseases. Every human being has a unique genetic code and there are many genetic variants within a group of individuals. Correspondingly, it may be difficult to identify which genes are likely to be of clinical interest in the context of a given genetic disease. By way of example, it may be difficult to detect and identify causal genes underlying the variant associations present within genome-wide association studies (GWAS).

With respect to such studies, genome-wide association studies (GWAS) have been used to identify thousands of genetic loci associated with various traits, such as complex traits and genetic diseases. Such studies may be useful for understanding processes and pathways associated with complex traits, including complex (e.g., multigenic) genetic diseases. However, for the majority of loci identified using GWAS the identity of the causal gene or genes that underly the association remains unclear or is hard to elucidate. Correspondingly, the biological insight that might be gained by such studies may be limited.

In particular, there are various challenges to identifying a causal gene via such studies. For example, linkage disequilibrium (LD) between variants may obfuscate the identity of the causal variant or of other biologically significant relationships. Further, many associated loci may not contain coding variants, but instead the causal variant may act through gene regulatory mechanisms. Incomplete maps from a regulatory element to a respective gene may therefore hinder causal gene identification.

In practice, such genetic variants will often be effectively neutral, with either no discernible difference in the expression of the encoded product or with a discernible difference in the expression of the encoded product, but where the difference has little or no phenotypic effect. Conversely, in other instances such genetic variations may be considered pathogenic, and may be associated with a negative phenotypic effect, such as a genetic-based disease or disorder. Though such pathogenic genetic variants have often been depleted from genomes by natural selection, rare variants especially have largely arisen in the human population recently without time for selection to act or the variant effect may not impact reproductive fitness. Thus, an ability to identify which genetic variants are likely to be pathogenic can facilitate the investigation of such genetic variants so as to gain an understanding of corresponding disease states, associated diagnostics, treatments, and/or cures.

### BRIEF DESCRIPTION

Systems, methods, and articles of manufacture are described for constructing a variant classifier (such as a pathogenicity classifier) and for using or refining such classifier information. Such implementations may include or utilize non-transitory computer readable storage medium storing instructions executable by a processor to perform actions of the system and methodology described herein. One or more features of an implementation can be combined with the base implementation or other implementations, even if not explicitly listed or described. Further, implementations that are not mutually exclusive are taught to be combinable such that one or more features of an implementation can be combined with other implementations. This disclosure periodically may remind the user of these options. However, omission from some implementations of recitations that repeat these options should not be taken as limiting the potential combinations taught in the following sections. Instead, these recitations are hereby incorporated forward by reference into each of the following implementations.

This system implementation and other systems disclosed optionally include some or all of the features as discussed herein. The system can also include features described in connection with methods disclosed. In the interest of conciseness, alternative combinations of system features are not individually enumerated. Further, features applicable to systems, methods, and articles of manufacture are not repeated for each statutory class set of base features. The reader will understand how features identified can readily be combined with base features in other statutory classes.

The present techniques provide for a software platform (e.g., a software application implemented locally (e.g., on-premise) or in a distributed (e.g., cloud implementation) manner and that provides tools for analyzing genetic data to identify variants of interest, including rare variants. In certain aspects, such a software platform may include one or more tools that allow a user to integrates multiple types of new and/or available data to derive useful variant pathogenicity or classification information. In certain embodiments, an output of the software platform may be a customized report and/or an updated or customized annotation file that includes relevant derived variant information for one or more genes. In practice, the software platform may be generic with respect to a sequencing device used to generate sequence data. Technical effects associated with the creation and use of such a customized report or annotation file include, but are not limited to, improved identification of functional variants, generation of improved datasets of rare variants that may be used in training or using machine learning tools or other models directed toward predicting gene expression effects of variants, pathogenicity of variants, and so forth.

With the preceding in mind, the present approach relates the development and use of machine learning (ML) (or other suitable or comparable artificial intelligence) methods to predict or identify causal genes from GWAS summary statistics. The disclosed ML approaches are useful in identifying causal genes and from such summary statistics and provide improved performance in terms of both precision (i.e., the ratio of true positives to declared positives (also referred to as the positive predictive value and corresponding to the complement of the false discovery rate) and recall (i.e., the number of true positive samples correctly classified by a model (also known as the true positive rate or sensitivity) compared to all other methods conventionally employed in the field. As disclosed herein, the presently described model and approaches perform well on both causal gene identification and gene prioritization within locus and allow estimation of p-values for evaluation.

As part of the presently described techniques, various gene-level associations may be computed from GWAS summary statistics (or other descriptive, comprehensive statistics) and may be used to learn enrichments of gene features derived from cell-type specific gene expression, biological pathways, and protein-protein interactions (PPI). In some embodiments a scoring system may be employed such that a score is assigned coding (or non-coding) regions and this score may be used to identify causal genes (e.g., genes associated with a genetic disease or disorder) for review or evaluation. Such scores and nominated regions may be presented to a reviewer, such as via a user interface of a processor-based system, and/or may be used as part of automatically selecting or recommending a diagnosis, treatment (e.g., pharmaceutical or biologic treatment), research target (e.g., drug research target), prognosis, or recommendation for an individual. In some embodiments, such selections or recommendations may be presented as a ranked list or with associated probabilistic assessments for consideration by a reviewer.

In one embodiment, a processor-implemented method is provided for detecting causal genes. In accordance with this method, a first set of values is generated by processing one or more variant-level features or sets of variant-level features using a first set of neural network layers. A second set of values is generated by processing one or more gene-level features or sets of gene-level features using a second set of neural network layers. The first set of values or embeddings derived from the first set of values and the second set of values or embeddings derived from the second set of values are processed using a third set of neural network layers. The third set of neural network layers generates a prediction score as an output. One or more causal genes are identified based on the prediction score. A drug or treatment is selected based upon the identified one or more causal genes.

In a further embodiment, one or more tangible, machine-readable media storing processor-executable routines are provided. In accordance with this embodiment, the processor-executable routines, when executed by a processor, cause acts to be performed comprising: generating a first set of values by processing one or more variant-level features or sets of variant-level features using a first set of neural network layers; generating a second set of values by processing one or more gene-level features or sets of gene-level features using a second set of neural network layers; processing the first set of values or embeddings derived from the first set of values and the second set of values or embeddings derived from the second set of values using a third set of neural network layers, wherein the third set of neural network layers generates a prediction score as an output; identifying one or more causal genes based on the prediction score; and selecting a drug or treatment based upon the identified one or more causal genes.

In an additional embodiment, a processor based system is provided. In accordance with this embodiment, the processor-based system comprises one or more processors configured to execute processor-executable code and one more memory or data storage structures storing processor-executable code. The processor-executable code, when executed by the one or more processors, causes the one or more processors to perform acts comprising: generating a first set of values by processing one or more variant-level features or sets of variant-level features using a first set of neural network layers; generating a second set of values by processing one or more gene-level features or sets of gene-level features using a second set of neural network layers; processing the first set of values or embeddings derived from the first set of values and the second set of values or embeddings derived from the second set of values using a third set of neural network layers, wherein the third set of neural network layers generates a prediction score as an output; identifying one or more causal genes based on the prediction score; and selecting a drug or treatment based upon the identified one or more causal genes.

### BRIEF DESCRIPTION OF DRAWINGS

These and other features, aspects, and advantages of the present invention will become better understood when the following detailed description is read with reference to the accompanying drawings in which like characters represent like parts throughout the drawings, wherein:

FIGS. 1A and 1B illustrates aspects of one embodiment of a machine learning pipeline for genome-wide association studies (GWAS), in accordance with aspects of the present techniques;

FIG. 2 illustrates aspects of a further embodiment of a machine learning pipeline for evaluation of GWAS, in accordance with aspects of the present techniques;

FIG. 3 illustrates further aspects of the embodiment of FIG. 2, in accordance with aspects of the present techniques;

FIG. 4 illustrates aspects of gradient boosting to combine deep learning (DL) model predictions with locus-based features, in accordance with aspects of the present techniques;

FIG. 5 illustrates a plot of precision-recall for the presently described model(s) with other techniques;

FIG. 6 illustrates a plot of model prediction scores for direct techniques versus random simulation techniques in the context of LDL cholesterol;

FIG. 7 depicts a schematic diagram of an implementation of a causal gene identification system in a networked or cloud computing environment, in accordance with aspects of the present technique; and

FIG. 8 is a simplified block diagram of a processor-based system, in accordance with aspects of the present technique.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

As used herein, the terms “application”, “pipeline”, “module”, “model”, “program”, and so forth may refer to one or more sets of computer software instructions (e.g., computer programs and/or scripts) executable by one or more processors of a computing system to provide particular functionality. Computer software instructions can be written in any suitable programming languages, such as C, C++, C #, Pascal, Fortran, Perl, Python, MATLAB, SAS, SPSS, JavaScript, AJAX, and JAVA. Such computer software instructions can comprise an independent or integrated application with data input and data display modules. Alternatively, the disclosed computer software instructions can be classes that are instantiated as distributed objects. The disclosed computer software instructions can also be component software. Additionally, the disclosed applications or engines can be implemented in computer software, computer hardware, or a combination thereof.

The detailed description of various implementations will be better understood when read in conjunction with the accompanying figures. To the extent that the figures illustrate concepts and functional blocks, modules, or processes, the depicted blocks or processes are not necessarily indicative of an implementation solely in hardware, software, or firmware. That is, such functionality may or may not be embodied in a discrete software module or modules, hardware circuit or circuits, and so forth, or indeed may be embodied or implemented in a combination of hardware and executable software. Further, applications, programs, modules, and/or computational pipelines as used herein may be implemented as a single executable unit (e.g., a stand-alone program) or may be implemented as a groups or series of separate or discrete such executable units, portions of which may operate in sequence or parallel or may operate when called upon as subroutines or callable modules. It should be understood that the various implementations are not limited to the arrangements and instrumentality shown in the figures, but may instead be implemented in other arrangements while providing the same or similar described functionality. In practice the functional units or blocks described in the figures may be implemented on different processors, computers, servers, or virtual machines and/or may be spread among local and/or remote execution platforms (e.g., a workstation, a local server, a cloud-based data center, and so forth).

As used herein, the term “computing system” or “processor-based system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the term “medium” refers to one or more non-transitory, computer-readable physical media that together store the contents described as being stored thereon. Embodiments may include non-volatile secondary storage, read-only memory (ROM), and/or random-access memory (RAM).

As discussed herein, the computational analysis of genomic data (e.g., sequence data) may be performed for the purpose of identifying variants of interest that may in turn be useful in identifying mechanisms of genetic disease as well as prospective treatments or pharmaceutical development. With this in mind, it may be useful to apply computational techniques, including techniques incorporating or based on artificial intelligence, to genetic and epigenetic studies so as to improve the performance of such techniques in determining genetic and regulatory associations with disease phenotypes.

As discussed herein, genome-wide association studies (GWAS) allow variants to be detected that are statistically significantly associated with disease risk. However, inferring which are the genes underlying these variant associations (i.e., the causal genes) is a challenging task. The techniques described herein substantially improve performance in terms of identifying such causal genes in terms of both precision (i.e., the ratio of true positives to declared positives (also referred to as the positive predictive value and corresponding to the complement of the false discovery rate) and recall (i.e., the number of true positive samples correctly classified by a model (also known as the true positive rate or sensitivity) compared to other approaches. For example, whereas prior or conventional approaches achieve accuracy and recall of less than 0.45 on the task of predicting a causal gene for each significant variant, the techniques described and disclosed herein achieves 0.65 on this task, providing >40% improvement.

As part of the presently described techniques various gene-level contributions or perturbations may be computed from GWAS summary statistics (or other available descriptive, comprehensive statistics) and may be used to learn or complement enrichments of gene features derived from other or parallel processing, such as cell-type specific gene expression, biological pathways (e.g., enriched pathways or networks), and/or protein-protein interactions (PPI). A score or prediction may be assigned according to these enrichments and this score may be used to nominate causal genes for review or evaluation. Such scores and nominated causal genes may be presented to a reviewer, such as via a user interface of a processor-based system, and/or may be used as part of automatically selecting or recommending a diagnosis, treatment (e.g., pharmaceutical or biologic treatment), research target, prognosis, or recommendation for an individual. In some embodiments, such selections or recommendations may be presented as a ranked list or with associated probabilistic assessments for consideration by a reviewer.

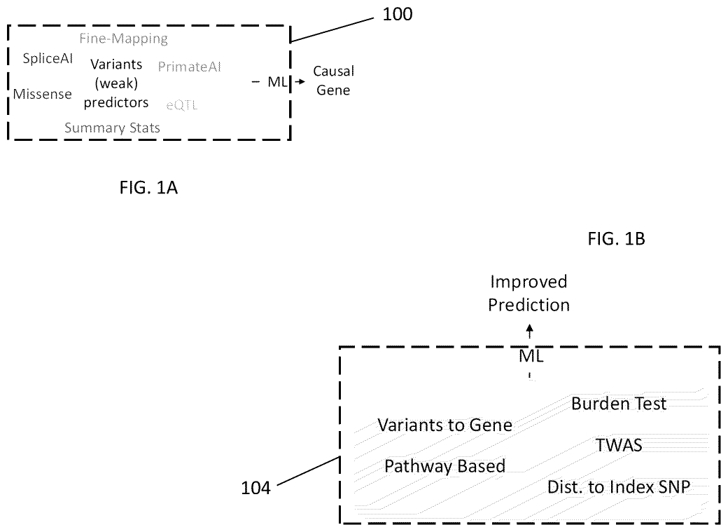

With the preceding in mind, and turning to the figures, FIGS. 1A and 1B illustrate aspects of one embodiment of a machine learning (ML) pipeline for identifying causal genes in GWAS. In one such embodiment, and as shown in FIG. 1A, a first part 100 or aspect of the model (or a separate first model in certain implementations) integrates variant level annotations to model variant-to-gene aspects of the data. Such integrated data may include, but is not limited to, GWAS summary statistics, fine mapping, artificial intelligence (AI) tools, loss of function data, missense data, expression quantitative trait loci (eQTL) data, and/or gene structure data. In the depicted example, and turning to FIG. 1B, a second part 104 or aspect may address other aspects of the data (such as, but not limited to, similarity-based data (e.g., polygenic priority score (PoPS) data), transcriptome-wide association study (TWAS), distance-to-index SNP data, metabolic pathway data, and so forth), such as to combine with other gene-level predictions. In practice, such a ML pipeline approach may be trained to predict thousands (e.g., ˜2,000) significant gene-trait associations across appropriate genome-wide association studies.

With this context in mind, the techniques discussed herein may be employed for processing genetic data (e.g., GWAS) to facilitate the detection of causal genes. Such processing may allow identification or derivation of causal genes and/or of metabolic pathway memberships associated with a disease or condition and, correspondingly, of potential treatments or remediations that may be employed in treating a disease.

It may be noted that in certain existing approaches to assessing GWAS data for the detection of causal genes, metrics such as MAGMA (Multi-marker Analysis of GenoMic Annotation) scores have been derived from an input (e.g., a summary statistics linkage disequilibrium (LD) reference panel) and employed as a dependent variable (e.g., “y”) in the analysis. Such approaches may be characterized as employing similarity-based methods to prioritize likely causal genes for assessment by searching for global patterns in associated genes and nominating those for consideration that have similar gene expression, functions, biological or metabolic pathway membership(s), and/or protein-protein interaction (PPI) network connections.

Certain embodiments of the techniques disclosed herein instead employ an estimated probability (e.g., the Improved Prediction indicated in FIG. 1B) that a given gene is the correct target (i.e., a causal gene of interest) as the dependent variable of the assessment. In certain such implementations, this probability is based on one or more of a distance (e.g., a ranked distance) to an index variant (e.g., an index single-nucleotide polymorphism (SNP)), biological or metabolic pathway data, transcriptome-wide association study (TWAS), and so forth. In certain such examples the estimated probabilities may be derived with reference to the summary statistics of a linkage disequilibrium (LD) reference panel generated from GWAS data.

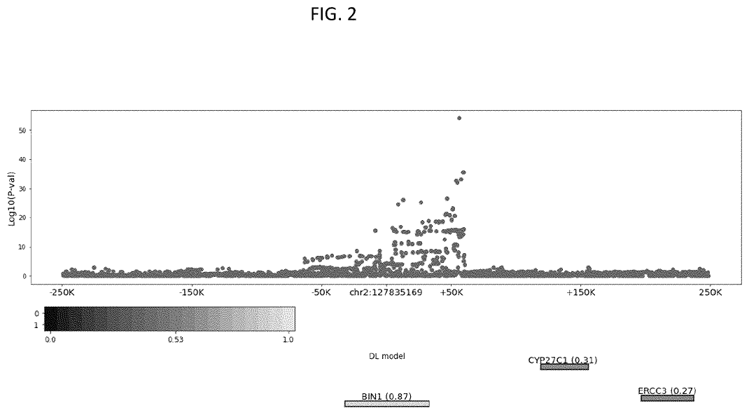

Turning to FIG. 2, further aspects of an implementation of such a ML pipeline are related and illustrated in which certain features are varied. The depicted example illustrates an embodiment in which the fine mapping window is enlarged (e.g., to 500 KB) and certain evaluated features are added or dropped. In the depicted plot, the resulting Log 10 (P-val) values (plotted on the y-axis) relative to the mapping window (500 KB) (plotted on the x-axis) may be used to identify a region of interest within the mapping window, here corresponding to BIN1 as evaluated using a deep learning (DL) model in the depicted example.

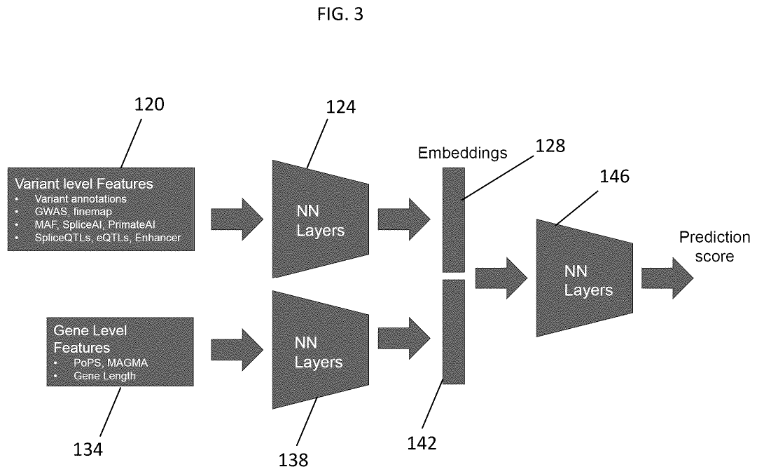

Turning to FIG. 3, further aspects of this approach are illustrated at a high-level in terms of a process-type flow. In this example, a combined deep learning (DL) model approach is illustrated. Along one branch of the combined DL model, variant level features 120 (e.g., variant annotations, GWAS, fine map, AI model outputs, eQTLs, and so forth) are provided as inputs to a first neural network 124 (depicted as NN layers) and embedding values 128 derived from the outputs. Similarly, along another branch of the combined DL model gene level features 134 (e.g., PoPS, Multi-marker Analysis of GenoMic Annotation (MAGMA), gene length, and so forth) are provided as inputs to a second neural network 138 (depicted as NN layers) (e.g., the same or a different neural network) and embedding values 142 derived from the outputs. The embeddings 128, 142 so derived from the respective neural networks 124, 138 (i.e., the respective variant level and gene level analyses) may themselves be provided as inputs to a third neural network 146 trained to output a prediction score that may be used for causal gene identification. In practice, identification of the causal gene in this manner may facilitate selection of a drug or treatment for a genetic disease associated with the identified causal gene.

More generally, and regarding machine-learning based predictors and classifiers, deep neural networks are a type of artificial neural networks that use multiple nonlinear and complex transforming layers to successively model high-level features. Deep neural networks provide feedback via backpropagation which carries the difference between observed and predicted output to adjust parameters. Deep neural networks have evolved with the availability of large training datasets, the power of parallel and distributed computing, and sophisticated training algorithms.

With respect to the neural networks, as discussed herein such networks may be used to implement certain of the analytics discussed herein, such as the generation of variant level analyses, gene level analyses, and causality prediction scores and the derivation of useful clinical analytics or metrics based on such scores or classifications. It should also be appreciated that, though neural networks are primarily discussed herein to provide a useful example and to facilitate explanation, other implementations may be employed in place of or in addition to neural network approaches, including but not limited to trained or suitably parameterized statistical models or techniques and/or other machine learning approaches. In particular, the following discussion may utilize certain concepts related to neural networks (e.g., convolutional neural networks) in implementations used to analyze certain genomic data of interest. With that in mind, certain aspects of the underlying biological and genetic problems of interest are outlined here to provide a useful context to the problems being addressed herein, for which the neural network techniques discussed herein may be utilized.

Convolutional neural networks (CNNs) and recurrent neural networks (RNNs) are components of deep neural networks. Convolutional neural networks may have an architecture that comprises convolution layers, nonlinear layers, and pooling layers. Recurrent neural networks are designed to utilize sequential information of input data with cyclic connections among building blocks like perceptrons, long short-term memory units, and gated recurrent units. In addition, many other emergent deep neural networks have been proposed for limited contexts, such as deep spatio-temporal neural networks, multi-dimensional recurrent neural networks, and convolutional auto-encoders. With respect to present application, given that sequence data are multi- and high-dimensional, deep neural networks have broad applicability and provide enhanced prediction power.

Training deep neural network involves optimizing the weight parameters in each layer, which gradually combine simpler features into complex features so that the most suitable hierarchical representations can be learned from data. A single cycle of the optimization process is organized as follows. First, given a training dataset, a forward pass sequentially computes the output in each layer and propagates the function signals forward through the neural network. In the final output layer, an objective loss function measures error between the inferenced outputs and the given labels. To minimize the training error, a backward pass uses the chain rule to backpropagate error signals and compute gradients with respect to all weights throughout the neural network. Finally, the weight parameters are updated using optimization algorithms based on stochastic gradient descent or other suitable approaches. Whereas batch gradient descent performs parameter updates for each complete dataset, stochastic gradient descent provides stochastic approximations by performing the updates for each small set of data examples. Several optimization algorithms stem from stochastic gradient descent.

Another element in the training of deep neural networks is regularization, which refers to strategies intended to avoid overfitting and thus achieve good generalization performance. For example, weight decay adds a penalty term to the objective loss function so that weight parameters converge to smaller absolute values. Dropout randomly removes hidden units from neural networks during training and can be considered an ensemble of possible subnetworks. Furthermore, batch normalization provides a new regularization method through normalization of scalar features for each activation within a mini-batch and learning each mean and variance as parameters.

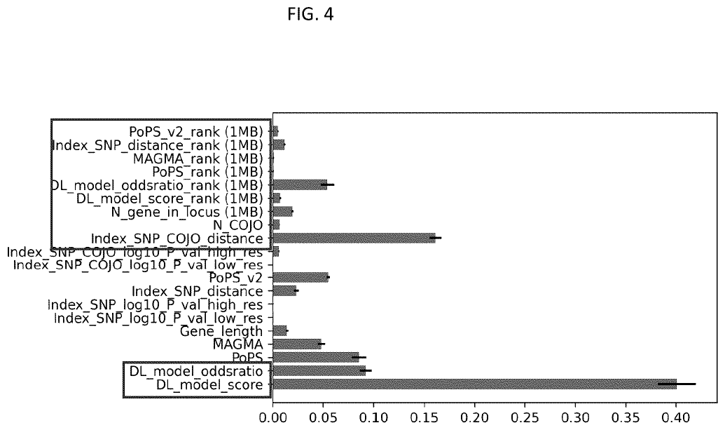

With the preceding in mind and turning to FIG. 4, a plot related to gradient boosting to combine deep learning model predictions with locus-based features is illustrated. The depicted plot depicts features arrayed along the y-axis, with relative scoring along the x-axis. As illustrated, the gradient boosted deep learning model approach as described herein performs well compared to other stand alone techniques and/or data sources.

With respect to this gradient boosting aspect, in practice a gradient-boosted machine learning approach may be employed to implement a classification and/or prediction framework as described herein. By way of example, XGBoost is one such machine learning technique that may be employed to implement a suitable classification and/or prediction framework. Such approaches may be implemented as decision tree ensemble learning algorithms suitable for classification processing techniques, where the ensemble learning algorithms combine multiple machine learning algorithms, as described herein, to improve model performance. In such contexts gradient-boosting may be understood to refer to improving the performance of a single, weak model by combination with other weak models so at to generate a collective model that is stronger than its constituents. In a gradient-boosted decision tree technique, therefore, an ensemble of shallow decision trees may be iteratively trained such that over each iteration error residuals of the previous model are used to fit the next model. A weighted sum of all of the decision tree predictions may be provide as the final prediction. With this in mind, in certain embodiments gradient boosting may be employed as part of the classification and probability estimation process.

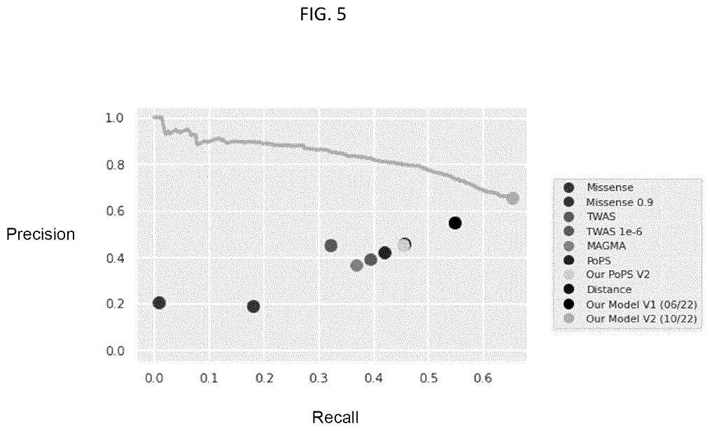

Turning to FIG. 5, a plot of precision (i.e., the ratio of true positives to declared positives (also referred to as the positive predictive value and corresponding to the complement of the false discovery rate) versus recall (i.e., the number of true positive samples correctly classified by a model (also known as the true positive rate or sensitivity) is illustrated for the presently described approaches (i.e., Model V1 and Model V2) along with other approaches or techniques. As may be observed the present approaches exhibit superior recall and precision relative to other techniques. By way of further illustration, the curve or plot of precision for different values of recall for a presently described model is shown, illustrating the tradeoffs that can be made in terms of recall to obtain a desired degree of precision (i.e., a greater degree of precision can generally be obtained with the presently described models at the expense of some degree of recall).

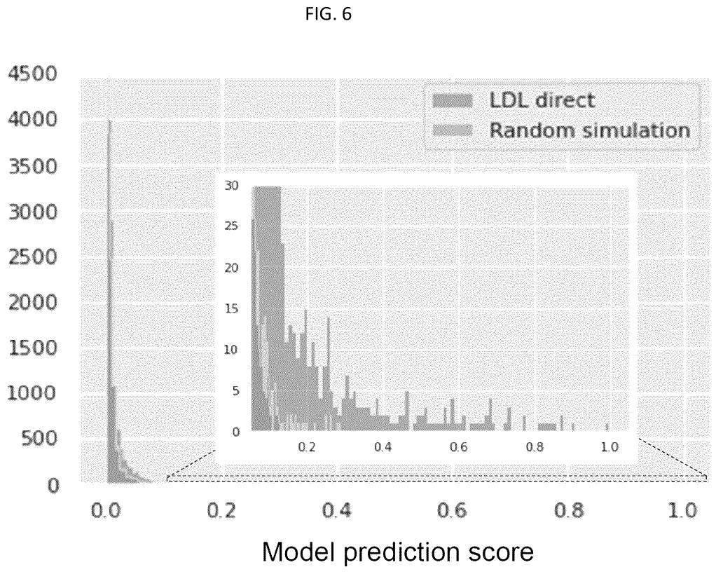

Turning to FIG. 6, a comparison of model prediction scores for work done on genes involved in cardiovascular events, including genes that modulate LDL cholesterol, are illustrated. In particular results for direct prediction approaches relative to random simulation is graphically depicted. The predicted value of the present techniques can be observed in this context, including the improvement relative to random simulation.

By way of summary, the presently described model performs well on both causal gene identification and gene prioritization within locus, allowing estimation of p-values that may be used for such identification and prioritization tasks. For certain embodiments, the area under the receiver-operating characteristic curves (AUC) for identifying causal genes may be 0.9. Further, gene prioritization within the window (e.g., a 1 MB window) may provide a top rank percentage of 0.65 (relative to 0.55 or lower obtained via pervious techniques) and may be within the top 5 rank percentage at a value of 0.90.

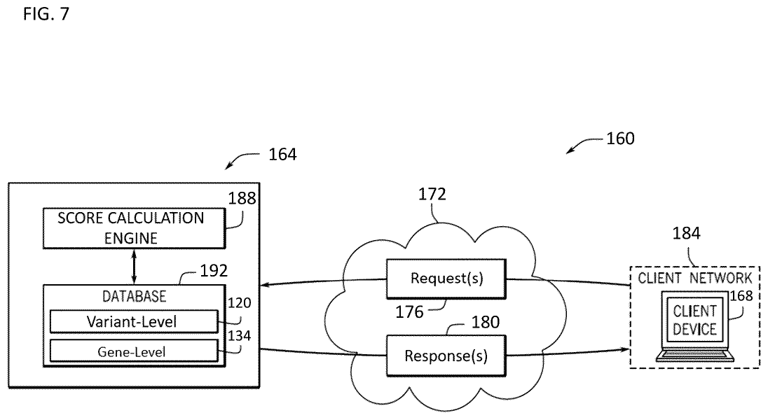

With the preceding in mind, and turning to FIG. 7, this figure illustrates aspects of one embodiment of a processor-based (e.g., computational) pipeline for identifying target (i.e., causal) genes in genetic data (e.g., GWAS data) as discussed herein. In particular, FIG. 7 depicts aspects of a cloud- or network-based approach by which variant-level features 120 and gene-level features 134 may be analyzed to derive one or more predictive scores that may be used to predict or prioritize potential target genes. In such a context, all or part of the analysis may occur remotely or, alternatively, the analysis may be performed using both local and remote resources. For example, certain aspects of the processes described herein may be performed at the datacenter or remote server, while other aspects of the processed may be performed locally at the workstation or thin-client. Further, though a cloud- or network-based approach is described with respect to FIG. 7 so as to provide a comprehensive example, in practice the processes and techniques described herein may be performed on a single processor-based device, either with or without a network connection. Thus, the example described with respect to FIG. 7 should be understood to not be limiting, but instead to provide context for one type of real-world implementation.

With this in mind, and turning to FIG. 7, a causal gene ranking or identification framework 160 is depicted in accordance with embodiments of the present technique. More specifically, FIG. 7 illustrates an abstraction of a cloud platform infrastructure and local client interface to the cloud infrastructure, such as via a local network. In this example, a cloud-based platform 164 (such as may be instantiated at a datacenter or a remote server) is connected to a client device 168 via a network 172 to facilitate processing of variant-level feature data 120 and gene-level feature data 134 in response to a request 176 to generate one or more responses 180 (e.g., prediction scores). Such a connection may be implemented via a web browser interface, a dedicated, standalone application, or other suitable program or data interfaces. In the depicted example, the client device 168 is itself part of or in communication with a local client network 184 that is configured to communicate with the network 172 that allows communication outside the client network 184. As used herein, a server, workstation, or other processor-based device may be understood to be implemented as a virtual instance (e.g., a virtual server) or as a physical or hardware implementation, though it should be understood that virtual servers also have underlying physical memory and processor aspects.

The implementation of the causal gene ranking or identification framework 160 illustrated in FIG. 7 includes a score calculation engine 188 configured to implement the logic and processes described herein and one or more databases 192 (either within a client instance, within the cloud-based platform 164 (e.g., within the datacenter or a related datacenter), or otherwise accessible by the instance and/or platform 164). The score calculation engine 188 may interact with a user of the client device 168 via requests 176 (e.g., requests to generate one or more predictive scores) and responses 180 (e.g., predictive scores).

For the embodiment illustrated in FIG. 7, the database 192 may be a stand-alone database, a database server instance, or a collection of database server instances. The illustrated database 192 may store or access GWAS data or panel results, one or more sources of expression data, one or more sources of interaction (e.g., protein-protein interaction (PPI) data, variant perturbation data, and/or one or more sources of pathway membership data, and so forth, as discussed herein. As discussed herein, the GWAS data and/or other data described may be used by the score calculation engine 180 to formulate a responsive reply 180, comprising a predictivity score or related metric or response, to the user of the client device 168.

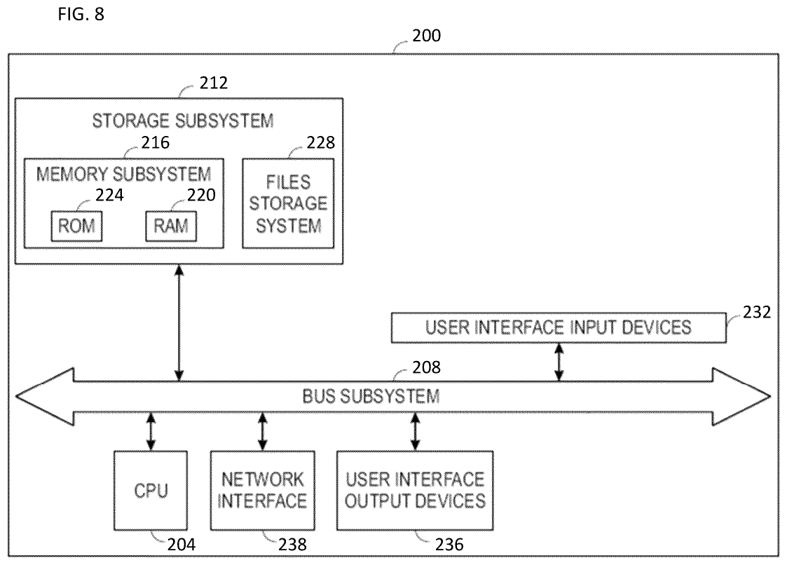

With the preceding in mind, FIG. 8 depicts an example of a processor-based system 200 (e.g., a workstation, a server, a thin client, a computer system, and so forth) suitable for use as the client device(s) 168 or as part of the cloud-based platform 164 in accordance with the framework illustrated in FIG. 7. In this example system, a high-level hardware architecture is described for reference. Such hardware may be physically embodied as one or more computer systems (e.g., servers, workstations, and so forth). It should be appreciated that the present example may include components not found in all embodiments of such a system or may not illustrate all components that may be found in such a system. Further, in practice aspects of the present approach may be implemented in part or entirely in a virtual server or client environment or as part of a cloud platform. However, in such contexts the various virtual server or client instantiations will still be implemented on an underlying hardware platform as described with respect to FIG. 8, although certain functional aspects described may be implemented at the level of the virtual server or client.

With this in mind, FIG. 8 is a simplified block diagram of a processor-based system (e.g., a computer system) 200 that can be used to implement the technology disclosed. Such a computer system typically includes at least one processor (e.g., microprocessor or CPU) 204 that communicates with a number of peripheral devices via bus subsystem 208. These peripheral devices can include a storage subsystem 212 including, for example, memory devices 216 (e.g., RAM 220 and ROM 224) and a file storage subsystem 228, user interface input devices 232, user interface output devices 236, and a network interface subsystem 238. The input and output devices allow user interaction with computer system (e.g., processing/storage systems). Network interface subsystem 238 provides an interface to outside networks, including an interface to corresponding interface devices in other computer systems.

In the context of the depicted processor-based system 200, the user interface input devices 232 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” may be construed as encompassing all possible types of devices and ways to input information into computer system.

User interface output devices 236 can include a display subsystem, a printer, a fax machine, or non-visual displays such as audio output devices. The display subsystem can include a cathode ray tube (CRT), a flat-panel device such as a liquid crystal display (LCD) or organic light emitting diode (OLED) display, a projection device, or some other mechanism for creating a visible image. The display subsystem can also provide a non-visual display such as audio output devices. In general, use of the term “output device” may be construed as encompassing all possible types of devices and ways to output information from the computer system to the user or to another machine or computer system.

Storage subsystem 212 stores programming and data constructs that provide the functionality of some or all of the modules and methods described herein, such as one or more of the score calculation engine 188, variant-level feature data 120 and gene-level feature data 134, neural networks or network layers 124, 138, 146 and so forth. Stored software modules are generally executed by a processor 204 alone or in combination with other processors 204. Data constructs or tables may be stored locally on the processor-based system 200 or accessed from a remote system on which they are stored in such a storage subsystem.

Memory 216 used in the storage subsystem 212 can include a number of memory structures or devices, such as a main random-access memory (RAM) 220 for storage of instructions and data during program execution and a read only memory (ROM) 224 in which fixed instructions are stored. A file storage subsystem 228 can provide persistent storage for program and data files, and can include a hard disk drive, solid state data drives, a floppy disk drive along with associated removable media, a CD-ROM drive, an optical drive, or removable media cartridges. The modules implementing the functionality of certain implementations can be stored by file storage subsystem 228 in the storage subsystem 212, or in other machines accessible by the processor 204.

Bus subsystem 208 provides a mechanism for letting the various components and subsystems of computer system communicate with each other. Although bus subsystem 208 is shown schematically as a single bus, alternative implementations of the bus subsystem 208 can use multiple busses.

The processor-based system 200 itself can be of varying types including a personal computer, a portable computer, a workstation, a computer terminal, a network computer, a thin client, a mainframe, a stand-alone server, a server farm, a widely-distributed set of loosely networked computers, or any other data processing system or user device. Due to the ever-changing nature of computers and networks, the description of a processor-based system 200 as depicted in FIG. 8 is intended only as an example for purposes of illustrating the functionality and types of components associated with the technology disclosed. Many other configurations of computer system are possible having more or less components or different components than the computer system depicted in FIG. 8.

With the preceding context in mind, the techniques discussed herein may be employed for processing genetic data (e.g., GWAS, variant-level feature data, gene-level feature data) to facilitate the detection of causal genes. Such processing may allow identification or derivation of causal genes and/or of metabolic pathway memberships associated with a disease or condition and, correspondingly, of potential treatments or remediations that may be employed in treating a disease.

While only certain features of the invention have been illustrated and described herein, many modifications and changes will occur to those skilled in the art. It is, therefore, to be understood that the appended claims are intended to cover all such modifications and changes as fall within the true spirit of the invention.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A processor-implemented method for detecting causal genes, comprising:
generating a first set of values by processing one or more variant-level features or sets of variant-level features using a first set of neural network layers;
generating a second set of values by processing one or more gene-level features or sets of gene-level features using a second set of neural network layers;
processing the first set of values or embeddings derived from the first set of values and the second set of values or embeddings derived from the second set of values using a third set of neural network layers, wherein the third set of neural network layers generates a prediction score as an output;
identifying one or more causal genes based on the prediction score; and
selecting a drug or treatment based upon the identified one or more causal genes.

2. The method of claim 1, wherein one or more of the first set of neural network layers, second set of neural network layers, or third set of neural network layers comprise a deep learning model.

3. The method of claim 1, wherein the one or more variant-level features comprise one or more of variant annotations, genome-wide association studies (GWAS) data, fine map data, AI model outputs, or expression quantitative trait loci (eQTL) data.

4. The method of claim 3, wherien the GWAS data comprises GWAS summary statistics.

5. The method of claim 1, wherein the one or more gene-level features comprise one or more of polygenic priority score (PoPS) data, multi-marker analysis of genomic annotation (MAGMA) data, or gene length data.

6. The method of claim 1, wherein the one or more causal genes, or variants of the one or more causal genes, are statistically significantly associated with disease risk.

7. One or more tangible, machine-readable media storing processor-executable routines, wherein the processor-executable routines, when executed by a processor, cause acts to be performed comprising:
generating a first set of values by processing one or more variant-level features or sets of variant-level features using a first set of neural network layers;
generating a second set of values by processing one or more gene-level features or sets of gene-level features using a second set of neural network layers;
processing the first set of values or embeddings derived from the first set of values and the second set of values or embeddings derived from the second set of values using a third set of neural network layers, wherein the third set of neural network layers generates a prediction score as an output;
identifying one or more causal genes based on the prediction score; and selecting a drug or treatment based upon the identified one or more causal genes.

8. The one or more tangible, machine-readable media of claim 7, wherein one or more of the first set of neural network layers, second set of neural network layers, or third set of neural network layers comprise a deep learning model.

9. The one or more tangible, machine-readable media of claim 7, wherein the one or more variant-level features comprise one or more of variant annotations, genome-wide association studies (GWAS) data, fine map data, AI model outputs, or expression quantitative trait loci (eQTL) data.

10. The one or more tangible, machine-readable media of claim 7, wherien the GWAS data comprises GWAS summary statistics.

11. The one or more tangible, machine-readable media of claim 7, wherein the one or more gene-level features comprise one or more of polygenic priority score (PoPS) data, multi-marker analysis of genomic annotation (MAGMA) data, or gene length data.

12. The one or more tangible, machine-readable media of claim 7, wherein the one or more causal genes, or variants of the one or more causal genes, are statistically significantly associated with disease risk.

13. A processor-based system, comprising:
one or more processors configured to execute processor-executable code; and
one more memory or data storage structures storing processor-executable code, which when executed by the one or more processors, causes the one or more processors to perform acts comprising:
generating a first set of values by processing one or more variant-level features or sets of variant-level features using a first set of neural network layers;
generating a second set of values by processing one or more gene-level features or sets of gene-level features using a second set of neural network layers;
processing the first set of values or embeddings derived from the first set of values and the second set of values or embeddings derived from the second set of values using a third set of neural network layers, wherein the third set of neural network layers generates a prediction score as an output;
identifying one or more causal genes based on the prediction score; and
selecting a drug or treatment based upon the identified one or more causal genes.

14. The processor-based system of claim 13, wherein one or more of the first set of neural network layers, second set of neural network layers, or third set of neural network layers comprise a deep learning model.

15. The processor-based system of claim 13, wherein the one or more variant-level features comprise one or more of variant annotations, genome-wide association studies (GWAS) data, fine map data, AI model outputs, or expression quantitative trait loci (eQTL) data.

16. The processor-based system of claim 13, wherein the GWAS data comprises GWAS summary statistics.

17. The processor-based system of claim 13, wherein the one or more gene-level features comprise one or more of polygenic priority score (PoPS) data, multi-marker analysis of genomic annotation (MAGMA) data, or gene length data.

18. The processor-based system of claim 13, wherein the one or more causal genes, or variants of the one or more causal genes, are statistically significantly associated with disease risk.
